(19) United States
(12) Plant Patent Application Publication
(10) Pub. No.: US 2004/0034895 P1
(43) Pub. Date: Feb. 19, 2004

(54) Chrysanthemum plant named Lemon Meringue

(76) Inventor: Nathan Q. Smith, Nipomo, CA (US)

Correspondence Address: BARBARA S. KITCHELL, SUITE 400, 222 LAKEVIEW AVENUE, WEST PALM BEACH, FL, 33401, US

(21) Appl. No.: 10/218,439

(22) Filed: Aug. 13, 2002

(51) Int. Cl. A01H 5/00

(52) U.S. Cl. PLT/295

(57) ABSTRACT

The Lemon Meringue is a new and distinct cultivar of chrysanthemum botanically known as Dendranthema grandiflora, and referred to by the cultivar name of Lemon Meringue.

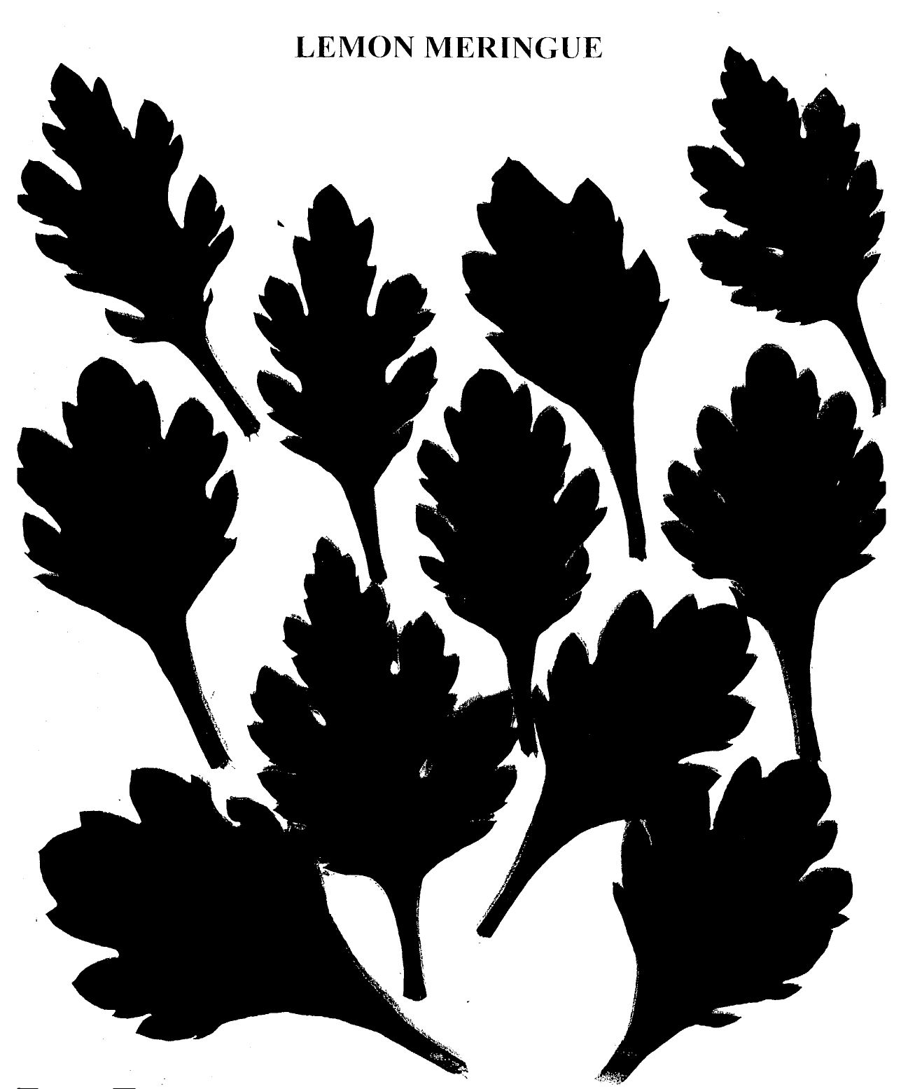

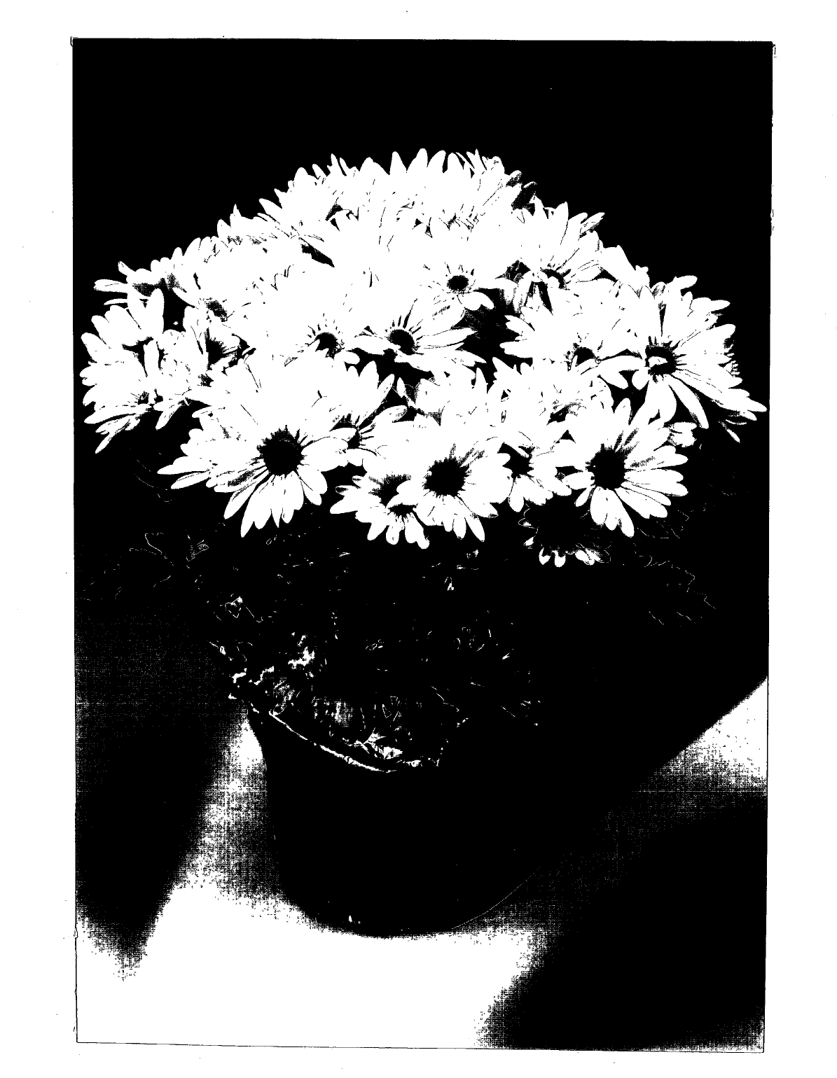

### BACKGROUND OF THE NEW PLANT

The female parent of lemon Meringue was the commercial variety Pink Blush, (U.S. Plant Pat. No. 9444) and the male parent was Icey isle, U.S. Plant Pat. No. 9416. The cross was made in the Spring of 1996.

Lemon meringue was discovered and selected as a unique flowering plant among the progeny of the stated cross by Nathan Q. Smith in September 1997 in a controlled environment at Clearwater Nursery, in Nipomo, Calif.

### SUMMARY OF THE INVENTION

Horticultural examination of controlled flowerings of successive plantings has shown that the unique combination of characteristics as herein disclosed for Lemon Meringue are firmly fixed and are retained through successive generations of asexual reproduction.

Lemon Meringue has not been observed under all possible environmental conditions. The phenotype may vary significantly with variations in the environment such as temperature, light intensity and day length.

The following observations, measurements and comparisons describe plants grown in a controlled environment in Nipomo, Calif. Unrooted cuttings were directly stuck into 6.5″ pots and grown under the natural temperature and day length prevailing in March through July of 2001. Lemon Meringue differs from parents Icey isle and Pink Blush, in the following ways:

1. It blooms in 8.5 weeks vs 8.0 weeks for both parents.

2. Its flower form is a daisy spoon vs. a daisy form on both parents.

3. It has a pronounced green daisy disc vs. a daisy form on both parents.

4. It can be produced year round vs. April through December for Icey Isle.

5. It produces a fuller more densely vegetative body than either parent.

6. It produces more flowers and branches than either parent.

7. Its ray color is yellow vs. Pink Blush with pink rays and Icey Isle with white rays.

8. Its foliage remains green throughout its life vs. Icey Isle's tendency to experience severe (although temporary) foliar chlorosis on the leaf just below the pinch during various periods of the year.

The following traits have been repeatedly observed and are determined to be basic characteristics Lemon Meringue, which in combination distinguish this chrysanthemum as a new and distinct cultivar.

1. Flat capitulum form.

2. Daisy capitulum type.

3. Clear bright yellow spoon formed outer ray florets.

4. Green daisy disc.

5. Diameter across the fact of the capitulum of 11.0 cm.

6. Exceptional upright to spreading habit with an average of 4.2 breaks after a single pinch.

7. Flowering response is approximately 60 days from the start of short days.

8. Relatively easy to control plant height from bench such that the finished height will range from 16 inches to 18 inches depending on pot size and market requirements.

### DESCRIPTION OF THE DRAWINGS

The accompanying drawings show a typical inflorescence and leaf characteristics of Lemon Meringue, with the colors being as nearly true as possible with illustrations of this type.

Sheet 1 is a color photograph Lemon Meringue grown as a pinched disbud pot mum with 5 cuttings in a 6.5″ pot.

Sheet 2 is a black and white photograph showing the under sides of mature leaves of Lemon Meringue.

Of the commercial cultivars known to the inventor, the most similar in form, color, and habit to Lemon Meringue is the cultivar Miramar. Reference is made to “Chart A” which compares certain characteristics of Lemon Meringue to the same characteristics of the aforementioned cultivar.

CHART A

| | YELLOW | LEMON | |
|---|---|---|---|
| CHARACTERISTIC | BLUSH | MERINGUE | MIRAMAR |
| CAPITULUM FORM | FLAT | FLAT | FLAT |
| CAPITULUM TYPE | DAISY | DAISY | DAISY |
| DIAMETER ACROSS | 8.5 CM | 8.5 CM | 8.7 CM |
| FACE OF THE | | | |
| CAPITULUM | | | |
| DIAMETER OF DISC | 1.8 CM | 1.7 CM | 1.9 CM |
| BRANCHING | UPRIGHT | UPRIGHT | UPRIGHT |
| PATTERN | AND | AND | AND |
| | SPREADING | SPREADING | SPREADING |
| BREAKS/ | 5 | 5 | 4 |
| PINCH | | | |
| HEIGHT IN A | 16-18″ | 16-18″ | 16-18″ |
| 6.5″ POT | | | |
| DISC FLORET | EDGE BEGIN | EDGE BEGIN | EDGE BEGIN |
| | W/RHS 9A | W/RHS 9A | W/RHS 9A |
| | GRADUAT- | GRADUAT- | GRADUAT- |
| | ING TO | ING TO | ING TO |
| | YELLOW | YELLOW | YELLOW |
| | GREEN RHS | GREEN RHS | GREEN RHS |
| | 144A | 144A | 144A |
| # DISC FLORETS | 190 | 243 | 348 |
| LENGTH OF DISC | 0.5 CM | 0.6 CM | 0.5 CM |
| FLORETS | | | |
| RAY FLORET | FRONT: | FRONT: | FRONT: |
| COLOR | GOLDEN | GOLDEN | GOLDEN |
| | YELLOW: | YELLOW: | YELLOW: |
| | RHS 5A | RHS 9A | RHS 5A |
| | BACK: | BACK: | BACK: |
| | PALE | PALE | PALE |
| | YELLOW: | YELLOW: | YELLOW: |
| | RHS 5B | RHS 8A | RHS 5C |
| # RAY FLORETS | 27.66 | 21 | 49 |
| SHAPE OF RAY | ELLIPTIC | ELLIPTIC/ | ELLIPTIC |
| FLORETS | | SPOON | |
| LENGTH OF RAY | 4.1 CM | 4.2 CM | 4.0 CM |
| FLORETS | | | |
| WIDTH OF RAY | 1.5 CM | 1.5 CM | 1.1 CM |
| FLORETS | | | |
| NUMBER OF | 8 | 7 | 7.2 |
| FLOWERS/BREAK | | | |
| LEAF COLOR | FRONT: | FRONT: | FRONT: |
| | RHS 147A | RHS 147A | RHS 147A |
| | BACK: | BACK: | BACK: |
| | RHS 147 B | RHS 147 B | RHS 147 B |
| MATURE LEAF | 5.6 CM | 6.6 CM | 6.1 CM |
| WIDTH | | | |
| MATURE LEAF | 9.6 CM | 10.8 CM | 10.9 CM |
| LENGTH | | | |
| RESPONSE/ | 8 WK | 8.5 WK | 9 WK |
| TREATMENT | | | |

Similar traits of the two cultivars include, the flat, daisy capitulum. The diameter across the face of the capitulum of Lemon Meringue is larger than the capitulum face diameter of Miramar. Both cultivars have similar upright, spreading branching patterns. Miramar has fewer breaks per pinch than Lemon Meringue. The finished plant height is similar for each cultivar. However, the disc floret color Lemon Meringue is prominently green, while Miramar yellow. Miramar tends to have more disc florets than Lemon Meringue. The length of the disc florets is greater for Lemon Meringue vs. Miramar. Using The ROYAL HORTICULTURAL SOCIETY COLOUR CHART as a standard reference, the front ray floret color for Lemon Meringue is Bright Yellow 5-A while the color of the front ray florets of Lemon Meringue is Pale Yellow 5-B. Miramar has more ray florets than Lemon Meringue. The ray florets of Lemon Meringue are spooned, while those of Miramar are elliptic. Lemon Meringue has ray florets that are longer in length than Miramar. The width of the ray florets of Lemon Meringue is equal to those of Miramar. The color of the leaves of Miramar is slightly darker green than the color of the leaves of Lemon Meringue. Miramar has longer and wider mature leaves than Lemon Meringue. Finally, Lemon Meringue has a response time of 8.5 weeks, while Miramar has 9.0 week response from the start of short days.

### DESCRIPTION OF THE NEW PLANT

### CLASSIFICATION

Botanical: Dendranthema grandiflora.

CV. name Lemon Meringue.

Commercial: Flat daisy spoon to be grown primarily as a DISBUD pot mum.

I. Inflorescence:

A. Capitulum

Form.—Flat.

Type.—Daisy.

Diameter across face.—average of 11.0 cm.

B. Corolla of Ray Florets

Color (general tonality from a distance of three meters).—Yellow.

Ray floret color.—Front Fully Expanded Ray Floret: RHS 5A Back Fully Expanded Ray Floret: RHS 5B.

C. Corolla of Disc Florets

Disc floret color.—Edge begins with Yellow RHS 9A to and quickly graduates to center Yellow-Green of RHS 144A.

D. Reproductive Organs

Androecium.—Present in disc florets only, pollen absent.

Gynoecium.—Present both in disc and ray florets.

II. Plant:

A. General Appearance

Height.—When grown in a 6.5″ pot with 1-4 applications of B-9 the total plant height, from the bottom of the pot may be maintained at 16 to 18 inches.

Branching pattern.—Upright and spreading with an average of 4.2 breaks when grown with one pinch.

B. Foliage:

Color (upper surface).—RHS 137A.

Color (lower surface).—RHS 137C.

Shape.—See Photograph.

What is claimed is:

1. A new and distinct chrysanthemum cultivar substantially as herein described and illustrated.
